(19) United States
(12) Patent Application Publication
(10) Pub. No.: US 2008/0139944 A1
(43) Pub. Date: Jun. 12, 2008

(54) Devices for covering ultrasound probes of ultrasound machines

(76) Inventors: Raymond F. Weymer, Philadelphia, PA (US); Michael G. Cannon, Haverford, PA (US); Lawrence A. Engle, Stowe, PA (US); Jodi Schwartz Klessel, Blue Bell, PA (US); Anthony P. Lannutti, Norristown, PA (US); Kevin S. Randall, Ambler, PA (US); Joseph A. Urbano, Audubon, PA (US)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 11/635,961

(22) Filed: Dec. 8, 2006

(51) Int. Cl. A61B 8/00

(52) U.S. Cl. 600/459; 600/437

(57) ABSTRACT

Preferred embodiments of devices for covering an ultrasound probe can include a membrane, such as a sheath, having one or more ultrasonic couplers attached thereto for transferring ultrasonic energy between the ultrasound probe and a body surface of a patient.

### FIELD OF THE INVENTION

The present invention relates generally to ultrasound imaging. More particularly, the invention relates to devices and methods for covering ultrasound probes of ultrasound machines, and acoustically coupling the ultrasound probes to body surfaces of a patient.

### BACKGROUND OF THE INVENTION

Ultrasound images are typically generated by placing an ultrasound probe of an ultrasound machine in contact with a body surface of a patient, and directing high-frequency acoustical energy at the body surface. The ultrasound probe senses the acoustical energy reflected from the body surface and the underlying portion of the patient's body. The reflected energy is converted to an electrical signal by the ultrasound probe. The signal is relayed to a processing and display unit of the ultrasound machine, where the signal is transformed into an image. The signal can be relayed by a cable connected to the ultrasound probe and the ultrasound machine, or by wireless means such as radio frequency (RF) transmission.

A water-based gel, commonly referred to as ultrasonic gel, is commonly used to facilitate transmission of acoustical energy between the ultrasound probe and the body surface. The ultrasonic gel facilitates transmission of acoustical energy by substantially eliminating gaps or pockets of air between the ultrasound probe and the body surface. Ultrasonic gel for use in sterile applications is usually supplied in a sealed packet that maintains the ultrasonic gel in a sterile condition prior to use.

Ultrasound procedures are often performed in a sterile environment. As the non-sterile ultrasound probe needs to be pressed against the body surface of the patient to obtain a satisfactory ultrasound image, the non-sterile ultrasound probe is typically covered with a sheath formed from a flexible, sterilizable, fluid-impermeable material such as latex, polyethylene, polyurethane, or other polymeric materials. Sterile ultrasonic gel is usually applied to the body surface, and to the interior surface of the sheath against which the ultrasound probe is pressed, to facilitate transmission of acoustical energy between the ultrasound probe and the body surface.

The ultrasonic gel within the sheath usually spreads beyond the sensing face of the ultrasound probe and covers other portions of the probe, due to the manipulation of the probe during the ultrasound procedure. In applications where a wired ultrasound probe is used, the ultrasonic gel may also spread to and cover the portion of the cable proximate the probe.

The relatively thick and slippery ultrasonic gel needs to be removed from the ultrasound probe (and its cable, if applicable) after the ultrasound procedure has been completed and before the next use of the ultrasound probe. Moreover, the body surface of the patient needs to be cleansed of the ultrasonic gel. The expenditures of time and effort associated with applying and removing the ultrasonic gel before and after the ultrasound procedure can adversely impact the workflow efficiency of the technician performing the procedure. Applying and removing the ultrasonic gel can cause patient discomfort when the body surface is sensitive from an injury, a surgical procedure, etc. Moreover, ultrasonic gel can spread into unwanted areas once applied.

Adequate supplies of sterile ultrasonic gel, and sterile wipes for the clean-up process need to be maintained during the ultrasound procedure, when the procedure is performed under sterile conditions. Also, the quality of the ultrasound image can be adversely affected when an excessive amount of ultrasonic gel is applied to the interior of the sheath or the body surface of the patient.

Applying the sheath to the ultrasound probe usually requires two individuals. In particular, a first individual who is properly prepped for a sterile environment is located within the sterile field, and holds the sheath at or near the perimeter of the sterile field. The first individual, and/or a second individual located outside of the sterile field apply sterile ultrasonic gel to the inside of the sheath. The second individual then places the non-sterile ultrasound probe inside the sheath without touching the exterior of the sheath. The first individual then closes and secures the open end of the sheath without touching the interior of the sheath or the ultrasound probe.

An ongoing need thus exists for devices and methods for acoustically coupling an ultrasound probe of an ultrasound machine with a body surface of a patient without the use of ultrasonic gel, while maintaining a sterile barrier between the ultrasound probe and the body surface.

### SUMMARY OF THE INVENTION

Preferred embodiments of devices for covering an ultrasound probe can include a membrane, such as a sheath, having one or more ultrasonic couplers attached thereto for transferring ultrasonic energy between the ultrasound probe and a body surface of a patient.

Preferred embodiments of devices for covering an ultrasound probe that generates and receives acoustical energy are provided. The devices can comprise a sheath that receives the ultrasound probe and is capable of forming a barrier between the ultrasound probe and a body surface toward which the acoustical energy is directed. The devices can also comprise an ultrasonic coupler attached to the sheath and capable of transferring the acoustical energy between the ultrasound probe and the body surface.

Preferred embodiments of devices for transferring acoustical energy comprise a membrane, and a first ultrasonic coupler in contact with a first side of the membrane. The devices also comprise a second ultrasonic coupler in contact with a second side of the membrane and aligned with the first ultrasonic coupler whereby the acoustical energy can be transferred between the first and second ultrasonic couplers by way of the membrane.

Preferred methods are provided for acoustically coupling an ultrasound probe and a body surface comprise inserting the ultrasound probe into a sheath, urging a sensing face of the ultrasound probe against an ultrasonic coupler attached to a sheath, and directing the ultrasonic energy though the ultrasonic coupler.

Preferred methods for preparing an ultrasound probe for use comprise covering a portion of the ultrasound probe with a sheath while the ultrasound probe is positioned on a device that holds the ultrasound probe, and grasping the portion of the ultrasound probe covered by the sheath. The methods also comprise lifting the probe from the device that holds the ultrasound probe, covering a remainder of the ultrasound probe with the sheath; and closing an open end of the sheath.

Preferred embodiments of devices for covering an ultrasound probe comprise a sheath that receives the ultrasound probe, a layer of ultrasonic gel disposed on an interior surface of the sheath, and a cover positioned over the ultrasonic gel and sealed to the interior surface of the sheath.

Preferred processes for supplying ultrasound probes for use in ultrasound procedures comprise providing the ultrasound probes to a user. Each of the ultrasound probes is packaged in a sheath that can form a fluid-impermeable barrier between the ultrasound probe and a body surface of a patient, and a sealed package that covers the sheath and the ultrasound probe and forms a barrier around the sheath and the ultrasound probe.

The processes also comprise receiving the ultrasound probes from the user after the ultrasound probes have been used in the ultrasound procedures, repackaging the ultrasound probes in new ones of the sheaths and the packages, and supplying the repackaged ultrasound probes to the user.

### BRIEF DESCRIPTION OF THE DRAWINGS

The foregoing summary, as well as the following detailed description of a preferred embodiment, are better understood when read in conjunction with the appended diagrammatic drawings. For the purpose of illustrating the invention, the drawings show an embodiment that is presently preferred. The invention is not limited, however, to the specific instrumentalities disclosed in the drawings. In the drawings:

FIG. 1 is a side view of a preferred embodiment of a device for covering an ultrasound probe, depicting the ultrasound probe about to be inserted into the device, with an end of the device in an open position;

FIG. 2 is a side view of the device and the ultrasound probe depicted in FIG. 1, showing the ultrasound probe fully inserted into the device with the end of the device in a closed position;

FIG. 3 is a magnified view of the area designated “A” in FIG. 1;

FIG. 4 is a side view of an alternative embodiment of the device shown in FIGS. 1-3, depicting the ultrasound probe shown in FIGS. 1 and 2 about to be inserted into the device, with an end of the device in an open position;

FIG. 5 is a bottom view of the device shown in FIG. 4;

FIG. 6 is a side view of another alternative embodiment of the device shown in FIGS. 1-3, depicting the ultrasound probe shown in FIGS. 1, 2, and 4 about to be inserted into the device, with an end of the device in an open position; and

FIG. 7 is a top view of the device shown in FIG. 6, with the end of the device in the open position.

### DETAILED DESCRIPTION OF ILLUSTRATIVE EMBODIMENTS

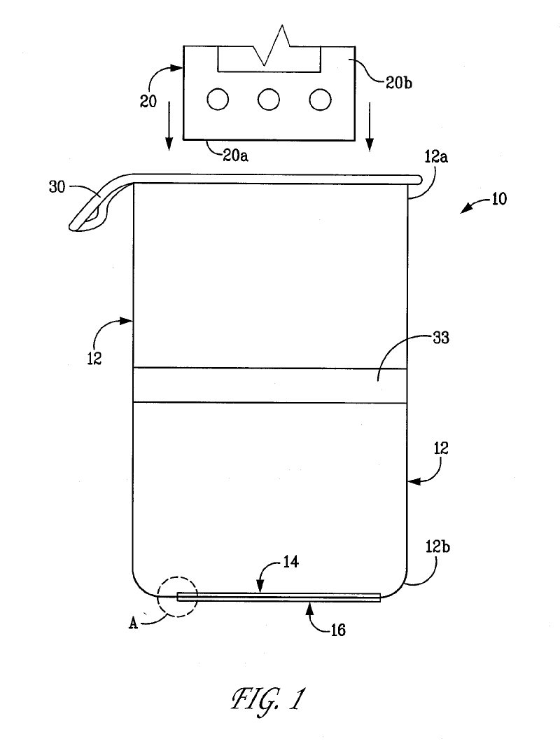

FIGS. 1-3 depict a preferred embodiment of a device 10 for covering an ultrasound probe 20 of an ultrasound machine. The device comprises a sheath 12, a first ultrasonic coupler 14, and a second ultrasonic coupler 16. The term “ultrasonic coupler,” as used throughout the specification and claims, refers to a device that transmits or transfers acoustical energy at ultrasonic frequencies, e.g., approximately 20 kHz to approximately 20 MHz or higher, with minimal attenuation. Typical imaging frequencies are in the range of approximately 1 MHz to approximately 20 MHz. An ultrasonic coupler can be formed from, for example, a liquid, a gel, or another substantially soft material, such as a pliable polymer matrix. Alternatively, an ultrasonic coupler can be formed from a water-containing polymer that is solidified by various methods.

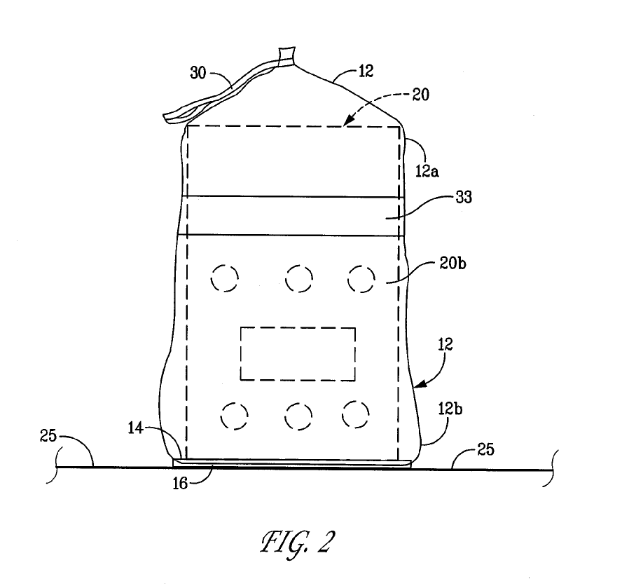

The sheath 12 receives the ultrasound probe 20, as shown in FIG. 2. The ultrasound probe 20 is a wireless ultrasound probe that communicates over a wireless link with a processing and display unit (not shown) of the ultrasound machine.

The use of the device 10 in conjunction with a wireless ultrasound probe is disclosed for exemplary purposes only. The device 10, and alternative embodiments thereof, can be used in conjunction with wired ultrasound probes.

The sheath 12 has an open end 12a that receives the ultrasound probe 20, as shown in FIG. 1. The device 10 can include a closure, such as a drawstring 30, to close the end 12a, and to maintain the end 12a in a closed condition after the ultrasound probe 20 has been inserted into the sheath 12, as shown in FIG. 2. Other types of closures can be used in the alternative. For example, the sheath 12 can have one or more bands of adhesive disposed thereon, proximate the end 12a. The adhesive can be covered by non-adhesive strips that can be removed by the user to expose the adhesive when the user wishes to secure the end of the sheath 12 in a closed condition. An elastic band can also be used as a closure in alternative embodiments.

In applications where the device 10 is used with a wired probe, the sheath 12 can be elongated so that the sheath 12 receives the entire wired probe, and a proximal portion of the probe's cable. The sheath 12 can include provisions, such as the above-mentioned drawstring 30 or adhesive bands, that permit the end 12a to be drawn around and secured to the wire.

The ultrasound probe 20 may be used in sterile environments. The sheath 12 therefore needs to function as a sterile barrier between the ultrasound probe 12, and the sterile environment. Accordingly, the sheath 12 should be formed from a flexible, sterilizable, fluid-impermeable material such as latex, polyethylene, polyurethane, or other suitable materials.

The first ultrasonic coupler 14 is attached to an interior, or inwardly-facing surface of the sheath 12. The second ultrasonic coupler 16 is attached to an exterior, or outwardly-facing surface of the sheath 12. The first and second ultrasonic couplers 14, 16 are aligned with each other as depicted in FIGS. 1-3. The first and second ultrasonic couplers 14, 16 are preferably located at an end 12b of the sheath 12 opposite the open end 12a, as shown in FIGS. 1 and 2. The end 12b receives a sensing face 20a of the ultrasound probe 20. The first and second ultrasonic couplers 14, 16 can be round, rectangular, or any other shape that is compatible with the sensing face 20a of the ultrasound probe 20.

The second ultrasonic coupler 16 can be positioned on a body surface 25 on the patient, and the sensing face 20a can be pressed against the first ultrasonic coupler 14 after the ultrasound transducer 20 has been placed in the sheath 12, as shown in FIG. 2. Acoustical energy generated by the ultrasound probe 20 and reflected from the tissue underlying the body surface 25 is transferred or conveyed between the ultrasound probe 20 and the body surface 25 by way of the first and second ultrasonic couplers 14, 16 and the underlying portion of the sheath 12. Because the user grasps the ultrasound probe 20 by way of the overlying sheath 12, the first ultrasonic coupler 14 can move with the ultrasound probe 20 and remain in contact with the sensing face 20a as the user moves the ultrasound probe 20 across the body surface 25 during the ultrasound procedure.

Each of the first and second ultrasonic couplers 14, 16 can comprise, for example, a pad 23 formed from a layer of cellulose material. The cellulose material can be impregnated with a mixture of glycerin and water.

Preferably, the outer periphery of each pad 23 is not impregnated with the mixture of glycerin and water. Adhesive can be applied to the outer periphery to secure the first or the second ultrasonic coupler 14, 16 to the sheath 12. The outer dimensions of each pad 23 are preferably greater than the outer dimensions of the sensing face 20a of the ultrasound probe 20, as shown in FIG. 2, so that the sensing face 20a can be aligned exclusively with the portion of the pad 23 that is impregnated with the mixture of glycerin and water.

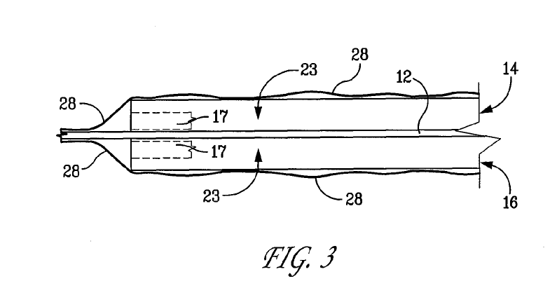

The first and second ultrasonic couplers 14, 16 can be attached to the sheath 12 by means other than adhesive in alternative embodiments. For example, the first and second ultrasonic couplers 14, 16 can each include a frame 17 (depicted in phantom in FIG. 3). The frame 17 can be formed from multiple pieces of rigid or semi-rigid material such as plastic. Alternatively, the frame 17 can be formed as a single continuous piece of the rigid or semi-rigid material configured as a rectangle, a ring, or another suitable geometric shape. The frame 17 can be glued to, or inserted within the outer peripheral edges of each pad 23, in lieu of placing adhesive on the outer peripheral edge. The frame 17 can be used to form a heat seal between the first and second ultrasonic couplers 14, 16 and the sheath 12. Alternatively, the frame 17 can be glued or otherwise attached to the sheath 12.

Specific details of the first and second ultrasonic couplers 14, 16 are provided for exemplary purposes only. Other types of ultrasonic couplers can be used in the alternative. For example, alternative embodiments of the first and second ultrasonic couplers 12, 14 can be formed from a gelled material, a part of which is hardened to facilitate attachment to the sheath 12. The gelled material can include, for example, a water-soluble polymeric compound that has been cross-linked by exposure to radiation or one or more freeze cycles. The polymeric compound can be polyvinyl alcohol, polyethylene oxide, polyacrylamide, or similar materials. A frame, such as the above-described frame 17, can be can be embedded in the hardened portion of the gelled material as the ultrasonic coupler is molded.

Other alternative embodiments of the first and second ultrasonic couplers 14, 16 can be formed as a sheet including a solid phase comprising a natural or synthetic hydrophilic block co-polymer and approximately 20 percent to approximately 95 percent by weight biocompatible liquid. For example, the first and second ultrasonic couplers 14, 16 can comprise a hydrophilic block co-polymer and greater than 70 percent by weight water or saline to form a flexible, solid hydrogel matrix. Other alternative embodiments of the first and second ultrasonic couplers 14, 16 can be formed, for example, from polyvinyl alcohol in combination with polyvinylpyrolidone, polyethylene glycols, and water.

The second ultrasonic coupler 16 should be sterile when the device 10 is used in a sterile application. The second ultrasonic coupler 16 does not need to be a sterile barrier, however, because the non-sterile ultrasound probe 20 is isolated from the sterile field and the body surface 25 by the sheath 12. The first ultrasonic coupler 14 does not have to be sterile when the device 10 is used in a sterile application, because the sheath 12 isolates the first ultrasonic coupler 14 from the sterile field and the body surface 25.

Preferably, the first and second ultrasonic couplers 14, 16 are each equipped with a cover 28. The covers 28 are shown only in FIG. 3, for clarity of illustration. Moreover, the side of each cover 28 is depicted as transparent in FIG. 3, to facilitate illustration of the underlying first and second ultrasonic couplers 14, 16.

The outer edges of each cover 28 are sealed to an underlying portion of the sheath 12 by a suitable means, such as a relatively weak adhesive, that permits the cover 28 to be removed by the user without damaging the sheath 12. Each cover 28 is preferably formed from a flexible, sterilizable, fluid-impermeable material that maintains the associated first or second ultrasonic coupler 14, 16 in a sterile condition, and prevents the first or second ultrasonic coupler 14, 16 from drying out prior to use. For example, each cover 28 can be formed from as a sheet of aluminum-alloy foil.

The device 10 can include a strap 33 attached to the sheath 12, as shown in FIGS. 1 and 2. The user's hand can be inserted between the strap 33 and underlying portion of the sheath 12, so that the strap 33 supports the ultrasound probe 20 from the user's hand. The use of the strap 33 can thus free one or more fingers of the user's hand to manipulate buttons, switches, or other operating features on the ultrasound probe 20. Other means for supporting the ultrasound probe 20 from the user's hand, such as individual loops for one or more of the user's fingers, can be used in the alternative.

The device 10 can facilitate the transmission of ultrasonic energy between an ultrasound probe, such as the ultrasound probe 20, and the body surface 25 on a patient without the use of ultrasonic gel. The use of the device 10 can thus improve workflow efficiency by eliminating the expenditures of time and effort associated with applying the ultrasonic gel to the body surface 25 and the ultrasound probe 20 before the ultrasound procedure, and cleaning the ultrasonic gel from the patient and the ultrasound probe 20 (and the cable of a wired ultrasound probe) after the procedure. Moreover, patient discomfort caused by applying and removing the ultrasonic gel to and from an injured or otherwise sensitive area can be eliminated through the use of the device 10.

A supply of sterile ultrasonic gel, and an supply of sterile wipes for clean-up do not need to be maintained when the device 10 is used in lieu of the ultrasonic gel. Also, the potential for degradation of the ultrasound image caused by applying a layer of ultrasonic gel that is too thick or too thin can be eliminated through the use of the device 10.

The device 10, and alternative embodiments thereof, can be installed on the ultrasound probe 20 by a single user in preparation for use in a sterile application, as follows. The user, properly prepped for the sterile-field environment, removes the device 10 from a sealed package or wrapping that maintains the device 10 in a sterile condition prior to use. The non-sterile ultrasound probe 20 is pre-positioned on a stand or other suitable holding device, with the sensing face 20a preferably facing generally upward and with most of the body 20b of the ultrasound probe 20 exposed. The stand can be located outside of the sterile field.

The user removes the cover 28 on the first ultrasonic coupler 14. The user then slides the sheath 12 over the sensing face 20a and a portion of the body 20b of the ultrasound probe 20, so that the first ultrasonic coupler contacts the sensing face 20a. The sheath 12 should be slid over the ultrasound probe 20 by the user using one or both hands held over the outside of the sheath 12, to avoid touching the non-sterile ultrasound probe 20.

The user can grasp the ultrasound probe 20 around the portion of the sheath 12 installed over the ultrasound probe 20 using one hand. The user can then lift and remove the ultrasound probe 20 from the stand. The user can pull the sheath 12 over the remainder of the ultrasound probe 12 using the other hand, once the ultrasound probe 20 is free of the stand. The end 12a of the sheath 12 can then be secured using the drawstring 30 or other closing means, so that the sheath 12 completely encloses the ultrasound probe 20. (In embodiments where the ultrasound probe is a wired probe, the sheath 12 can be extended up the probe's cable.) The cover 28 on the second ultrasonic coupler 16 can be removed to place the ultrasound probe 20 and the device 10 in a ready-to-use condition. The user can thus install the device 10 on the non-sterile ultrasound probe 20 while remaining isolated from the ultrasound probe 20, and without assistance from another individual.

Wireless ultrasound probes such as the ultrasound probe 20 can be supplied to a health care provider already covered in the device 10 or alternative embodiments thereof, in a condition ready for use in an ultrasound procedure. The device 10 and the enclosed ultrasound probe 20 can be placed in a hermetically-sealed package that acts as a fluid-impermeable barrier, to maintain the exterior surfaces of the device 10 in a sterile condition prior to use. After use, the device 10 can be discarded, and the ultrasound probe 20 can be returned to the supplier for repackaging in a new device 10. Alternatively, a hospital, clinic, doctor's office, or like establishment can establish internal facilities and processes for packaging devices 10 and ultrasound probes 20 in hermetically-sealed packages, and providing the ready-to-use devices 10 and ultrasound probes 20 to operators at the hospital, clinic, or doctor's office.

The supplier can maintain ownership of the ultrasound probes 20. The supplier can supply the pre-packaged ultrasound probes 20 on a pre-arranged schedule, upon return of the used ultrasound probes 20, or in accordance with other arrangements between the supplier and the user. A predetermined fee can be charged to the user for each time an ultrasound probe is provided by the supplier. Other payment arrangements, such as a fixed fee for a predetermined number of pre-packaged ultrasound probes 20, can be used in the alternative.

The foregoing description is provided for the purpose of explanation and is not to be construed as limiting the invention. Although the invention has been described with reference to preferred embodiments or preferred methods, it is understood that the words which have been used herein are words of description and illustration, rather than words of limitation. Furthermore, although the invention has been described herein with reference to particular structure, methods, and embodiments, the invention is not intended to be limited to the particulars disclosed herein, as the invention extends to all structures, methods and uses that are within the scope of the appended claims. Those skilled in the relevant art, having the benefit of the teachings of this specification, may effect numerous modifications to the invention as described herein, and changes may be made without departing from the scope and spirit of the invention as defined by the appended claims.

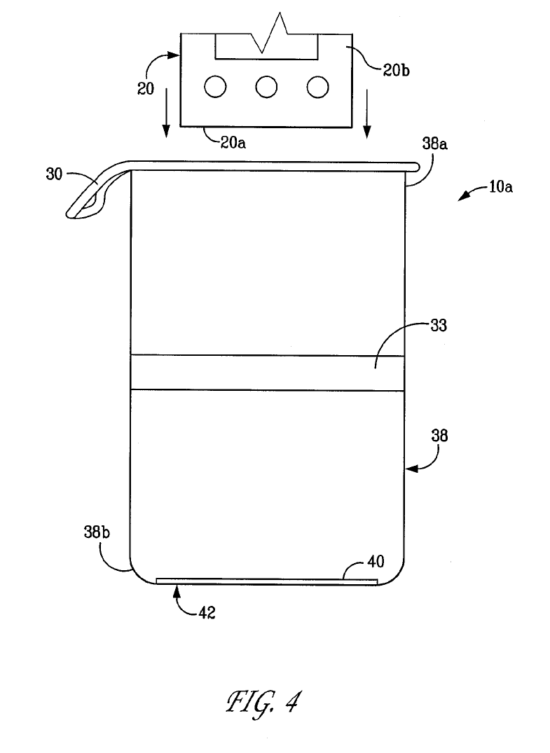

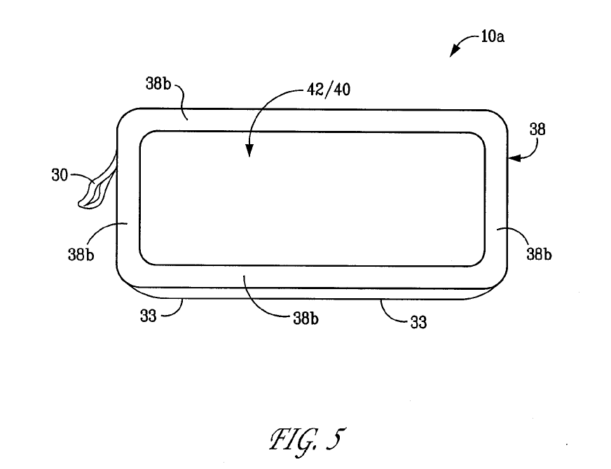

Alternative embodiments of device 10 can include a single ultrasonic coupler. For example, FIGS. 4 and 5 depict a cover device 10a comprising a sheath 38, and an ultrasonic coupler 40 positioned within the sheath 38. The sheath 38 can be substantially identical to the sheath 12, with the exception that the sheath 38 has an opening 42 formed in an end 38b thereof to provide access to the ultrasonic coupler 40. The outer dimensions of the ultrasonic coupler 40 should be greater than the corresponding dimensions of the opening 42. This configuration causes the outer edges of the ultrasonic coupler 40 to overlap the portion of the sheath 12 proximate the opening 42, while the remainder of the ultrasonic coupler 40 spans the opening 42.

The outer edges of the ultrasonic coupler 40 can be sealed to the overlapping surfaces of the sheath 38 by a suitable means such as adhesive. Alternatively, the outer edges can have a plastic piece or pieces attached thereto or embedded therein, as described above in relation to the first and second ultrasonic couplers 14, 16. The plastic piece or pieces can be sealed to the overlapping surfaces of the sheath 38 by glue, a heat seal, or other suitable means. The seal needs to be continuous, to maintain a sterile barrier between the non-sterile ultrasound probe 20 and the body surface 25 during use.

A cover, such as the cover 28 of the device 10, can be positioned over each side of the ultrasonic coupler 40. The cover 28 is not shown in FIG. 4 or 5, for clarity of illustration. The device 10a can include a strap 33 attached to the sheath 38, as discussed above in relation to the device 10.

In use, the transducer 20 is placed in the sheath 38 by way of an open end 38a thereof, after the cover 28 positioned on the inwardly-facing side of the ultrasonic coupler 40 has been removed by the user. The open end 38a can be closed and secured as described above in relation to the sheath 12. The cover 28 positioned on the outwardly-facing side of the ultrasonic coupler 40 can be removed to place the ultrasound probe 20 and the device 10a in a ready-to-use condition.

The sensing face 20a of the ultrasound probe 20 can be pressed against the inwardly-facing surface of the ultrasonic coupler 40, and the outwardly-facing surface of the ultrasonic coupler 40 can be placed against the body surface 25 of the patient. Ultrasonic energy can then be transferred between the sensing face 20a and the body surface 25 by way of the ultrasonic coupler 40.

The ultrasonic coupler 40 should act as a sterile barrier when the device 10 is used in a sterile application, as the ultrasonic coupler 40 is the only structure between the ultrasound probe 20 and the body surface 25. Accordingly, the ultrasonic coupler 40 should be formed from at least one layer of solid, fluid-impermeable material when the device 10a is to be used in a sterile application. For example, the ultrasonic coupler 40 can be formed from a water-soluble polymeric compound that has been cross-linked by radiation. The polymeric compound can be polyvinyl alcohol, polyethylene oxide, polyacrylamide, or similar materials.

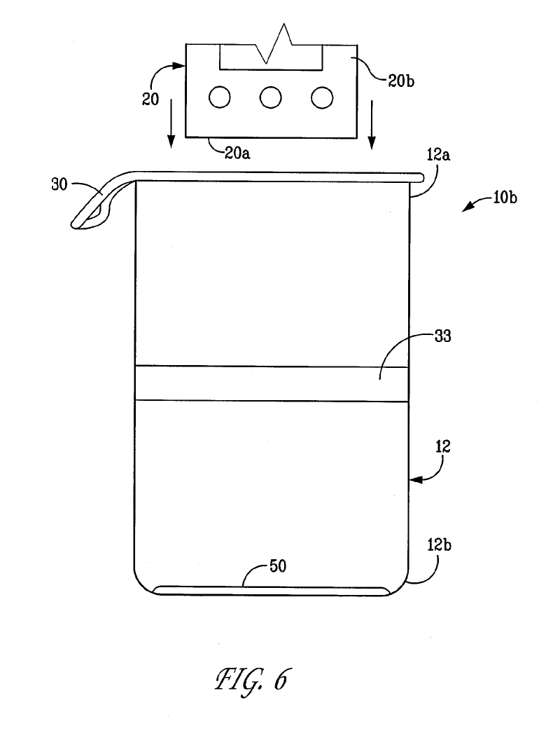

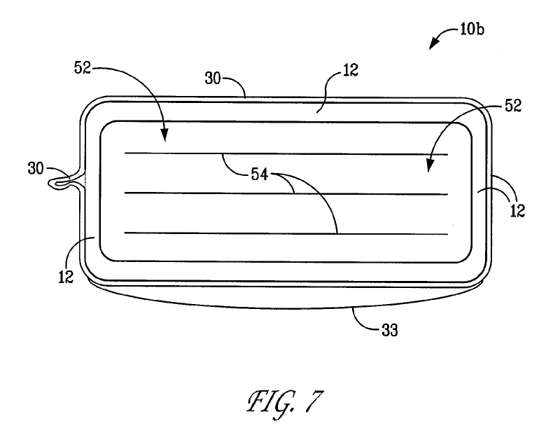

FIGS. 6 and 7 depict another alternative embodiment in the form of a cover device 10b. The device 10b comprises a sheath such as the sheath 12 described above in relation to the device 10. The device 10b also includes a layer of ultrasonic gel 50 disposed on an interior surface of the sheath 12, at the second end 12b thereof.

The device 10b further comprises a cover 52 positioned over the layer of ultrasonic gel 50. The outer edge of the cover 52 can be sealed to an underlying portion of the sheath 12 by a suitable means, such as a relatively weak adhesive, that permits the cover 52 to be removed by the user without damaging the sheath 12. The cover 52 is not depicted in FIG. 6, for clarity of illustration.

The cover 52 can be removed from the sheath 12 to expose the ultrasonic gel 50, prior to insertion of the ultrasound probe 20 in the sheath 12. Alternatively, the cover 52 can be configured with a weakened area formed by score lines 54, perforations, or other suitable means, as shown in FIG. 7. The weakened area can rupture relatively easily when the sensing face 20a is pressed against the cover 52. The sensing face 20a can be coated with the ultrasonic gel 50 by moving the sensing face 20a back and forth across the cover 52, while pressing the sensing face 20a against the cover 28 to squeeze the ultrasonic gel 50 through the ruptured area in the cover 28. The body surface 25 can be coated with additional ultrasonic gel, to acoustically couple the outer surface of the sheath 12 and the body surface 25.

The use of the device 10b does not eliminate the need to clean ultrasonic gel from the ultrasound probe 20 or the body surface 25 after the ultrasound procedure. The time and effort associated with applying ultrasonic gel to the interior surface of the sheath 12, however, can be eliminated through the use of the device 10b.

What is claimed is:

1. A device for covering an ultrasound probe that generates and receives acoustical energy, the device comprising:
a sheath that receives the ultrasound probe and is capable of forming a barrier between the ultrasound probe and a body surface toward which the acoustical energy is directed; and
an ultrasonic coupler attached to the sheath and capable of transferring the acoustical energy between the ultrasound probe and the body surface.

2. The device of claim 1, further comprising a second ultrasonic coupler, wherein the ultrasonic couplers are attached to opposite sides of the sheath and are aligned with each other whereby the acoustical energy can be transferred between the ultrasound probe and the body surface by way of the ultrasonic couplers and the sheath.

3. The device of claim 1, wherein the sheath has an opening formed therein, and the ultrasonic coupler spans the opening whereby a first side of the ultrasonic coupler can be placed against the body surface while the ultrasound probe is placed against a second side of the ultrasonic coupler.

4. The device of claim 3, wherein an outer peripheral edge of the ultrasonic coupler is attached to an edge of the sheath proximate the opening.

5. The device of claim 1, wherein the ultrasonic coupler comprises a pad formed from cellulose, and at least a portion of the pad is impregnated with a mixture of glycerin and water.

6. The device of claim 5, wherein an outer peripheral edge of the pad is substantially free of the mixture of glycerin and water, and the outer peripheral edge of the pad is attached to the sheath.

7. The device of claim 1, wherein the ultrasonic coupler comprises a water-containing polymeric gel.

8. The device of claim 1, wherein the ultrasonic coupler comprises a hydrophilic block co-polymer and an approximately 20 percent to approximately 95 percent by weight biocompatible liquid.

9. The device of claim 8, wherein the ultrasonic coupler comprises a hydrophilic block co-polymer and an approximately 70 percent higher by weight biocompatible liquid.

10. The device of claim 1, wherein the ultrasonic coupler comprises a frame formed from a rigid or semi-rigid material.

11. The device of claim 10, wherein the ultrasonic coupler further comprises a gelled material, wherein at least a portion of the gelled material is hardened.

12. The device of claim 11, wherein the frame is integrally formed with the hardened portion of the gelled material.

13. The device of claim 11, wherein the frame is molded into the hardened portion of the gelled material.

14. The device of claim 11, wherein the gelled material is a water-containing polymeric compound.

15. The device of claim 10, wherein the frame is attached to the sheath by a heat seal or an adhesive.

16. The device of claim 1, further comprising a removable cover positioned over the ultrasonic coupler.

17. The device of claim 1, wherein the ultrasonic coupler is attached to a first end of the sheath, and the device further comprises a closure that closes a second end of the sheath.

18. The device of claim 17, wherein the closure is a drawstring, an adhesive strip, or an elastic band.

19. The device of claim 1, further comprising a strap attached to the sheath for securing the device to a hand of a user.

20. The device of claim 1, wherein the sheath is formed from a fluid-impermeable material.

21. The device of claim 20, wherein the fluid-impermeable material is flexible and sterilizable.

22. A system, comprising the device of claim 1, and a sterile barrier, wherein the device is sealed within the sterile barrier.

23. The system of claim 22, wherein the sterile barrier is a package.

24. The system of claim 22, further comprising the ultrasound probe, wherein the ultrasound probe is disposed within the sheath.

25. The system of claim 24, wherein the ultrasound probe is wireless.

26. A device for transferring acoustical energy, comprising:
a membrane;
a first ultrasonic coupler in contact with a first side of the membrane; and
a second ultrasonic coupler in contact with a second side of the membrane and aligned with the first ultrasonic coupler whereby the acoustical energy can be transferred between the first and second ultrasonic couplers by way of the membrane.

27. The device of claim 26, wherein the membrane is a sheath that receives an ultrasound probe that generates the acoustical energy.

28. The device of claim 27, wherein the sheath is formed from a fluid-impermeable material.

29. The device of claim 28, wherein the fluid-impermeable material is flexible and sterilizable.

30. The device of claim 26, wherein the first ultrasonic coupler is attached to the first side of the membrane by adhesive applied to an outer peripheral edge of the first ultrasonic coupler, and the second ultrasonic coupler is attached to the second side of the membrane by adhesive applied to an outer peripheral edge of the second ultrasonic coupler.

31. The device of claim 30, wherein:
the first ultrasonic coupler comprises a pad formed from cellulose, and a portion of the pad inward of the outer peripheral edge of the first ultrasonic coupler is impregnated with a mixture of glycerin and water; and
the second ultrasonic coupler comprises a pad formed from cellulose, and a portion of the pad of the second ultrasonic coupler inward of the outer peripheral edge of the second ultrasonic coupler is impregnated with the mixture of glycerin and water.

32. The device of claim 26, wherein:
the first ultrasonic coupler comprises a frame formed from a rigid or semi-rigid material, and the frame is attached to the first side of the membrane; and
the second ultrasonic coupler comprises a frame formed from the rigid or semi-rigid material, and the frame of the second ultrasonic coupler is attached to the second side of the membrane.

33. The device of claim 27, wherein the first and second ultrasonic couplers are attached to a first end of the sheath, and the device further comprises means for closing a second end of the sheath.

34. The device of claim 27, further comprising a strap attached to the sheath for securing the device to a hand of a user.

35. The device of claim 26, wherein the first and second ultrasonic couplers comprise a water-containing polymeric compound.

36. The device of claim 26, wherein the first and second ultrasonic couplers comprise a hydrophilic block co-polymer and an approximately 20 percent to approximately 95 percent by weight biocompatible liquid.

37. The device of claim 36, wherein the first and second ultrasonic coupler comprises a hydrophilic block co-polymer and an approximately 70 percent higher by weight biocompatible liquid.

38. A system, comprising the device of claim 27, and a sterile barrier, wherein the device is sealed within the sterile barrier.

39. The system of claim 38, wherein the sterile barrier is a package.

40. The system of claim 38, further comprising the ultrasound probe, wherein the ultrasound probe is disposed within the sheath.

41. The system of claim 40, wherein the ultrasound probe is wireless.

42. A method for acoustically coupling an ultrasound probe and a body surface, the method comprising:
inserting the ultrasound probe into a sheath;
urging a sensing face of the ultrasound probe against an ultrasonic coupler attached to a sheath; and
directing the ultrasonic energy though the ultrasonic coupler.

43. The method of claim 42, further comprising urging another ultrasonic coupler attached to the sheath against the body surface and directing the ultrasonic energy though the ultrasonic couplers and the sheath.

44. The method of claim 43, wherein urging a sensing face of the ultrasound probe against an ultrasonic coupler attached to a sheath comprises urging the ultrasound probe against a first side of the ultrasonic coupler and urging a second side of the ultrasonic coupler against the body surface.

45. The method of claim 42, further comprising closing an end of the sheath through which the ultrasound probe is inserted, and securing the end in a closed position.

46. The method of claim 42, further comprising supporting the ultrasound probe by way of a strap attached to the sheath.

47. The method of claim 42, further comprising removing a cover disposed over the ultrasonic coupler before urging the sensing face of the ultrasound probe against the ultrasonic coupler.

48. The method of claim 42, wherein the ultrasound probe is wireless.

49. A method for preparing an ultrasound probe for use, comprising:
covering a portion of the ultrasound probe with a sheath while the ultrasound probe is positioned on a device that holds the ultrasound probe;
grasping the portion of the ultrasound probe covered by the sheath;
lifting the probe from the device that holds the ultrasound probe;
covering a remainder of the ultrasound probe with the sheath; and
closing an open end of the sheath.

50. The method of claim 49, wherein the device that holds the ultrasound probe is a stand.

51. The method of claim 49, wherein the device that holds the ultrasound probe is located outside of a sterile field.

52. The method of claim 51, further comprising moving the ultrasound probe into the sterile field after closing the open end of the sheath.

53. The method of claim 49, wherein lifting the ultrasound probe from the stand comprises lifting the ultrasound probe from the stand using one hand of the user; and covering a remainder of the ultrasound probe with the sheath comprises covering a remainder of the ultrasound probe with the sheath using another hand of the user.

54. A device for covering an ultrasound probe, the device comprising:
a sheath that receives the ultrasound probe;
a layer of ultrasonic gel disposed on an interior surface of the sheath; and
a cover positioned over the ultrasonic gel and sealed to the interior surface of the sheath.

55. The device of claim 54, wherein the cover is removably attached to the interior surface of the sheath.

56. The device of claim 54, wherein the cover has a weakened area formed therein that can rupture when the ultrasound probe is pressed against the cover.

57. A process for supplying ultrasound probes for use in ultrasound procedures, comprising:
providing the ultrasound probes to a user, with each of the ultrasound probes being packaged in (i) a sheath that can form a sterile, fluid-impermeable barrier between the ultrasound probe and a body surface of a patient, and (ii) a sealed package that covers the sheath and the ultrasound probe and forms a barrier around the sheath and the ultrasound probe;
receiving the ultrasound probes from the user after the ultrasound probes have been used in the ultrasound procedures;
repackaging the ultrasound probes in new ones of the sheaths and the packages; and
supplying the repackaged ultrasound probes to the user.
